(19) United States
(12) Patent Application Publication
(10) Pub. No.: US 2009/0088709 A1
(43) Pub. Date: Apr. 2, 2009

(54) Illuminated fluid collection bag

(76) Inventor: Lawrence A. Salvadori, San Diego, CA (US)

Correspondence Address: Tyco Healthcare Group LP;d/b/a Covidien, 15 Hampshire Street, Mansfield, MA, 02048, US

(21) Appl. No.: 11/904,567

(22) Filed: Sep. 27, 2007

(51) Int. Cl. A61M 1/00

(52) U.S. Cl. 604/317

(57) ABSTRACT

A urine collection bag is provided which includes a light source to allow a caregiver to take fluid readings during sleep periods, and other lights-out periods, without needing to turn on room lighting and risk disturbing the patient. The light can be permanently mounted to the bag and discarded with the bag after use. A reusable light is also provided which can be moved from bag to bag. Embodiments are presented wherein the light attaches to a bag specifically accommodated to accept lighting, as well as embodiments where the light can be used with any commonly-available collection or distribution bag.

### BACKGROUND

1. Technical Field

The present disclosure relates to fluid collection bags for receiving bodily fluids. More specifically, the present disclosure relates to a urine collection bag and urine metering device which includes a self-powered light for enabling a patient caregiver to obtain volumetric readings under low ambient lighting conditions.

2. Background of Related Art

Fluid collection systems for collecting bodily fluids such as urine are well known in the art. Typically, urine collection systems include a fluid collection bag defining a fluid chamber and having an inlet port or ports for receiving fluid and a discharge port to facilitate drainage of the collection bag. A discharge tube can be attached to the discharge port and a discharge valve is provided to regulate fluid flow, e.g., drainage, from the collection bag. The bag typically includes graduation marks to enable an observer to determine the volume of fluid contained within the bag.

In use, a urine collection bag is typically hung on a support structure such as a bed frame, positioned below a catheterized patient. A drain tube having one end connected to the catheterized patient and a second end in fluid communication with the urine collection bag directs urine to the urine collection bag. As urine is collected, it is necessary for the patient caregiver to periodically record the visual properties and volume of fluid collected from the patient. However, during sleeping periods, the room lights are generally turned off, which presents a challenge to the caregiver. In this instance, the caregiver must either turn on the room lights, which can disturb the sleeping patient; introduce a flashlight or other portable illumination means into the patient care area, which can be awkward and pose a risk of cross-infection; or attempt to read the bag under suboptimal lighting conditions, which can lead to inaccurate measurements being recorded.

Similarly, fluid distribution systems such as intravenous drip bags also require periodic inspection to monitor the volume of fluid remaining in the bag, which during “lights-off” hours places the same burdens on a caretaker.

Accordingly, it would be desirable to provide a fluid collection bag and/or a fluid distribution bag that enables the patient caregiver to take readings under low light conditions without requiring the use of room lights or other external light sources.

### SUMMARY

In accordance with the present disclosure, a fluid collection bag having a light source for illuminating the collected fluid is presented. In an embodiment, the light source is an assembly having a battery-operated light-emitting diode (LED) and a pushbutton switch for activating the LED. The light is positioned on the bag to illuminate the volumetric graduations of the bag thereby enabling the patient caregiver to obtain accurate readings, and to permit visual assessment of the collected fluid. When inadequate ambient light is available to read the bag (during night rounds, for example) the caregiver presses the pushbutton to illuminate the bag and/or its contents so that readings can be taken without disturbing the patient or requiring the caregiver to carry and manipulate a flashlight.

It is envisioned the pushbutton switch operates the light in a momentary-contact mode, whereby the light is illuminated only while the button is depressed. Additionally or alternatively, it is envisioned the pushbutton operates in a push-on/push-off mode, whereby pushing the button alternately toggles the light on and off. In another embodiment, a single press of the pushbutton causes the light to turn on for a fixed period of time, for example, ten seconds, after which the light self-extinguishes. Other embodiments are contemplated wherein the light is extinguished if the pushbutton is pressed before the timeout period elapses. In this embodiment, the pushbutton switch operates in a primarily push-on/push-off mode with a battery-conserving timeout feature.

In an embodiment according to the present disclosure, the light assembly includes at least one power source, such as a button-type lithium battery, an LED coupled in series to a current-limiting resistor as will be familiar to the skilled artisan, and a normally open single-pole, single-throw (SPST) switch operatively coupled to a pushbutton. Pressing the pushbutton causes the switch to close which completes an electrical circuit, which in turn causes current to flow from the at least one battery through the resistor and LED, which activated the LED and illuminates the bag and its contents.

In an alternative embodiment, the light assembly includes a controller which accepts input from the pushbutton switch and activates the LED in accordance with a control algorithm. The control algorithm can cause the light to operate in push-on/push-off mode, push-on/push-off mode with timeout, momentary mode (i.e. push on/release off). Other functions are contemplated, such as pulse-width modulation of the LED activation current, to dim or fade the light; or an alarm state which flashes the LED in response to, for example, a “low battery” condition, a “bag full” condition, or a “bag empty” condition. An embodiment is envisioned wherein the light assembly includes at least one sensor for causing a bag full condition or a bag empty condition to be sensed by the controller, thus triggering the appropriate “bag full” or “bag empty” alarm state.

It is a further aspect of the present disclosure that the light assembly be substantially permanently mounted to the bag such that the bag and light assembly comprise a single disposable unit. For example, the disclosed light assembly can be affixed to the bag by adhesive, heat welding, lamination, or other suitable means. Also contemplated is a light that is removably mounted to the bag by clip-on means, by clamping means, by insertion into a holder on the bag dimensioned to receive and retain the light, by semi-permanent adhesive means, or by other suitable means. It is further envisioned the light can be used with bags particularly configured to accommodate the light, or with existing bags having no specific accommodations for the light.

In another envisioned embodiment, a fluid distribution bag, such as an intravenous (IV) drip bag, having a light source for illuminating the contents of the bag is provided.

In yet another embodiment contemplated by the present disclosure, a light dispersion shroud is provided which shields the caretaker's eyes from glare, and ideally, directs light towards the bag and its contents.

### BRIEF DESCRIPTION OF THE DRAWINGS

Various embodiments of the presently disclosed fluid collection bag with light are disclosed herein with reference to the drawings, wherein:

FIG. 1 is a front view of a first embodiment of a fluid collection bag having a light for illuminating the collected fluid in accordance with the present disclosure;

FIG. 2 is a front view of a second embodiment of a light for illuminating the collected fluid in a fluid collection bag in accordance with the present disclosure;

FIG. 3 is a front cross section view of a second embodiment of a light for illuminating the collected fluid in a fluid collection bag in accordance with the present disclosure;

FIG. 4 is a bottom view of a pushbutton corresponding to a second embodiment of a light for illuminating the collected fluid in a fluid collection bag in accordance with the present disclosure;

FIG. 5A is a side cross section view of a second embodiment of the disclosed light in a deactivated state;

FIG. 5B is a side cross section view of a second embodiment of the disclosed light in an activated state;

FIG. 6A is a side cross section view of a third embodiment of the disclosed light in a deactivated state;

FIG. 6B is a side cross section view of a third embodiment of the disclosed light in an activated state;

FIG. 7A is a side view of another embodiment of a light for illuminating the collected fluid in a fluid collection bag in accordance with the present disclosure;

FIG. 7B is a side view of yet another embodiment of a light having a light dispersion shroud for illuminating the collected fluid in a fluid collection bag in accordance with the present disclosure;

FIG. 8A is a schematic diagram illustrating a light for illuminating the collected fluid in a fluid collection bag in accordance with the present disclosure;

FIG. 8A is a schematic diagram illustrating a light having a controller for illuminating the collected fluid in a fluid collection bag in accordance with the present disclosure;

FIG. 9A is a cross-sectional view of a light having a controller for illuminating the collected fluid in a fluid collection bag that is substantially permanently mounted to the bag in accordance with the present disclosure;

FIG. 9B is a cross-sectional view of a light having a controller for illuminating the collected fluid in a fluid collection bag that is substantially permanently mounted to the bag in accordance with the present disclosure;

FIG. 9C is a cross-sectional view of a light having a controller for illuminating the collected fluid in a fluid collection bag that is removably mounted to the bag in accordance with the present disclosure;

FIG. 10A is a front view of a light having a controller for illuminating the collected fluid in a fluid collection bag with clip mounting means in accordance with the present disclosure;

FIG. 10B is a side view of a light having a controller for illuminating the collected fluid in a fluid collection bag with clamp mounting means in accordance with the present disclosure; and

FIG. 10C is a view of a light having a controller for illuminating the collected fluid in a fluid collection bag removably mounted by clamping means to the bag in accordance with the present disclosure.

### DETAILED DESCRIPTION OF EMBODIMENTS

Embodiments of the presently disclosed fluid collection bag assembly with light will now be described in detail with reference to the drawings wherein like reference numerals designate identical or corresponding elements in each of the several views.

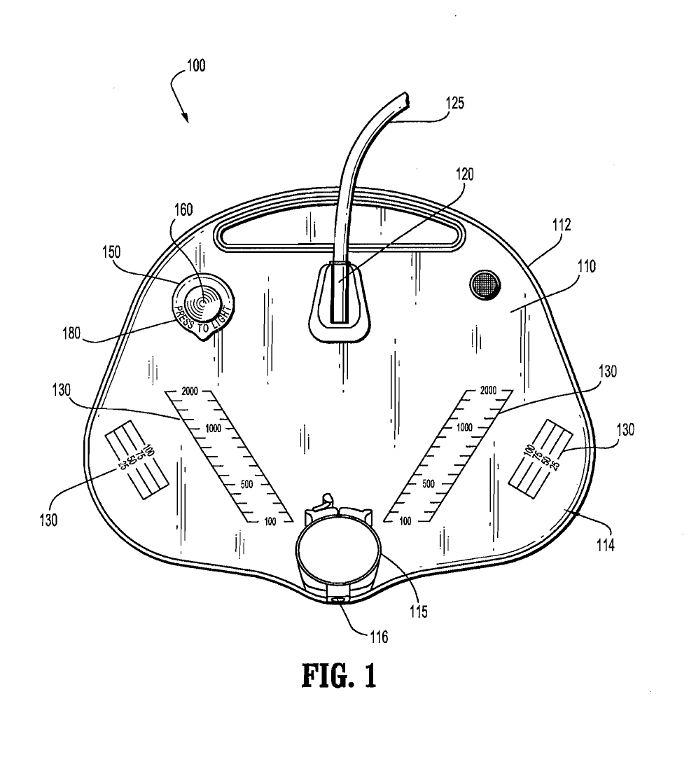

In FIG. 1 is shown an embodiment of a fluid collection bag 100 in accordance with the present disclosure constructed from a front panel 110 and a rear panel (not shown) which are sealably joined at their edges 112 by welding, gluing, or other suitable means to define a fluid chamber 114. Front panel 110 and rear panel can be constructed from polyvinyl chloride or other suitable flexible material. Preferably, front panel 100 is substantially transparent to enable an observer to view the contents of the fluid chamber. Graduations 130 for measuring the volume of fluid contained in fluid chamber 114 are imprinted on front panel 110 by silkscreen transfer, offset printing, heat printing or other suitable printing means. To provide an uncluttered field of observation and to enhance readability, rear panel (not shown) is preferably substantially opaque and preferably contrasting in color to that of graduations 130. Collection bag 100 further includes discharge valve 115 for emptying the fluid chamber of fluid through discharge port 116, and inlet port 120 which is dimensioned to accept inlet tube 125 to direct fluid from a catheterized patient (not shown) into fluid chamber 114.

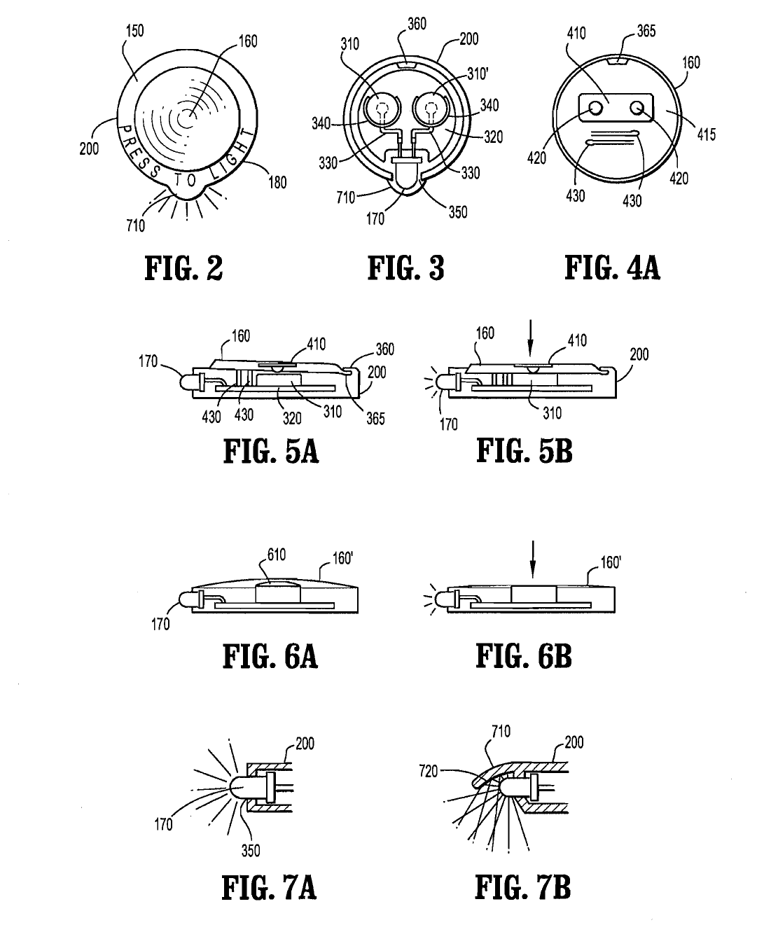

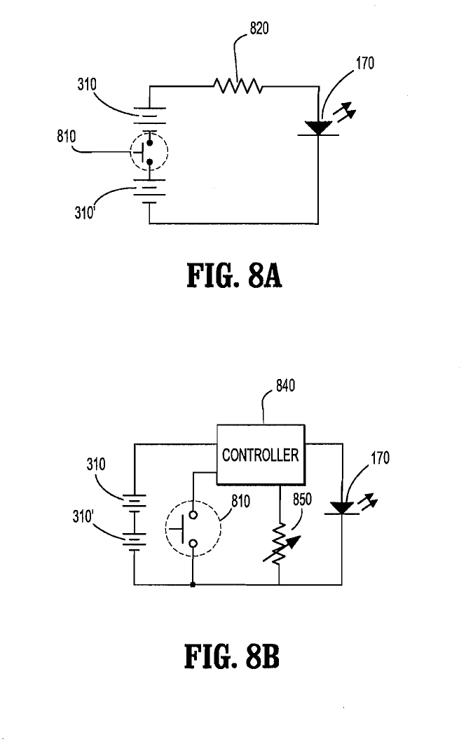

Disposed upon front panel 110 is light assembly 150. Referring now to FIGS. 2, 3, and 4, light assembly 150 includes a pushbutton 160 that is hingibly mounted to housing 200 by interlocking hinge members 360 and 365. Preferably, pushbutton 160 and housing 200 are constructed of electrically non-conductive material. Suitable materials for pushbutton 160 and housing 200 include without limitation such plastics as polypropylene, polystyrene, ABS, PVC or other suitable polymeric material. Circuit board 320 is fixedly disposed within housing 200 by adhesive, clip, heat welding or other suitable means. Circuit board 320 includes LED 170, preferably mounted thereto by soldering, and batteries 310, 310′ mounted thereto by soldering, battery clip 340 or other means known in the art. Preferably, the at least one battery 310, 310′ can be a small form factor device, such as a lithium, alkaline, or silver oxide button cell battery. LED 170 and batteries 310, 310′ are coupled by circuit traces 330 in accordance with FIG. 8A wherein anode of LED 170 is coupled to the positive terminal of battery 310, and cathode of LED 170 is coupled to the negative terminal of battery 310′. The negative terminal of battery 310 and positive terminal of battery 310′, each facing generally away from circuit board 320, comprise switch contacts as will be further described hereinbelow. It will be appreciated that embodiments which include a single battery, or greater than two batteries, or configurations with opposite polarities, are also contemplated within the scope of the present disclosure. The circuit optionally includes current-limiting resistor 820 having a value selected in accordance with the combined voltage of batteries 310, 310′ and the nominal forward bias current of LED 170 as is well-known. Preferably, current-limiting resistor 820 is included within LED 170, or, alternatively, current-limiting resistor 820 can be separately included, for example, on circuit board 320.

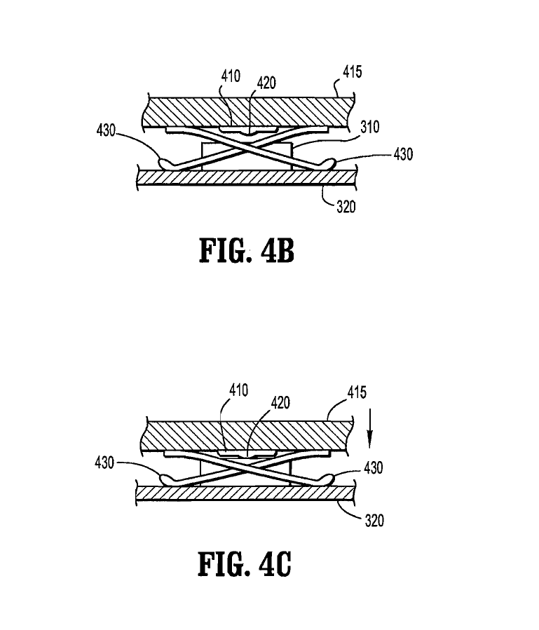

Turning to FIGS. 4A-C, pushbutton 160 includes switch wiper 410 having outwardly-dimpled contacts 420 disposed on pushbutton inner surface 415 positioned in substantial alignment with the centerline of batteries 310, 310′. Leaf springs 430 extend from inner surface 415 to circuit board 320 to bias pushbutton 160 away from batteries 310, 310′, causing pushbutton 160 to rest in the inactive position as shown in FIGS. 4B and 5A. Turning to FIGS. 4C and 5B, the light is activated when pushbutton 160 is depressed causing contacts 420 to electrically couple with the switch contacts comprising negative terminal of battery 310 and positive terminal of battery 310′, completing an electrical circuit and causing LED 170 to illuminate. Upon release of pushbutton 160, springs 430 restore pushbutton 160 to its inactive position causing contacts 420 to separate from batteries 310, 310′ breaking the circuit thus causing LED 170 to extinguish.

Referring now to FIGS. 6A and 6B, an embodiment is shown that includes resilient pushbutton 160′, having a dome-like shape, for activating switch 610, which can be a snap dome switch, membrane switch, or other suitable switch for activating LED 170. The application of external pressure, i.e. finger pressure, causes resilient pushbutton 160′ to deform, placing it in operative communication with and thereby actuating switch 610, causing LED 170 to activate. Upon the removal of external pressure, resilient pushbutton 160′ assumes its original shape, allowing switch 610 to deactivate.

With reference to FIGS. 7A and 7B, LED 170 protrudes through aperture 350 from housing 200 to illuminate the fluid collection bag. In an embodiment, LED 170 is sealably coupled to aperture 350 by suitable means to prevent fluid infiltration into housing 150. Preferably, LED 170 has a wide dispersion angle. In an embodiment, light 150 includes light dispersion shroud 710 to reduce glare by shielding direct rays of LED 170 from the observer's eyes. Additionally or optionally, inner surface 720 of light dispersion shroud 710 is contoured to reflect output of LED 170 generally towards fluid fluid chamber 114 and graduations 130. Preferably LED 170 is of the type which emits white light, however, the use of single- or multi-colored LEDs is contemplated wherein, for example, pushbutton actuation of the light causes LED 170 to emit white light while an alarm state causes LED 170 to emit steady and/or blinking colored light.

In yet another embodiment contemplated by the present disclosure, light 150 includes controller 840 configured for controlling LED 170 as shown in FIG. 8B. Controller receives user input from switch means 810, such as switch 610, that is operatively coupled to pushbutton 160 or 160′. Additionally or optionally, controller 840 receives input from fluid sensor 850, which is in communication with fluid chamber 114. Additionally or optionally, controller includes an undervoltage detection circuit (not shown) as well-known in the art for measuring the combined voltage of the at least one battery 310. Upon receiving a user input, i.e., a momentary button press, from switch means 810, controller 840 causes LED 170 to be illuminated for a predetermined time period, for example, ten seconds, whereupon controller 840 causes LED 170 to extinguish at the expiration of the timeout period. Additionally, a second user input received prior to expiration of the timeout period can also cause extinguishment of LED 170. In yet another embodiment, controller 840 causes LED 170 to remain illuminated as long as a continuous user input is received, i.e., a continuous button press, and to extinguish upon termination of the continuous user input, i.e., button release. In an embodiment, controller 840 can determine whether an alarm state exists and in response thereto cause LED 170 to illuminate in a continuous or blinking fashion. An alarm state can be caused by, for example, input received by controller 840 from fluid sensor 850 indicating a bag full condition, a bag empty condition, or from the undervoltage detection circuit within controller 840 indicating the combined voltage of the at least one battery 310 is below an acceptable threshold, i.e. the at least one battery 310 has reached the end of its useful life.

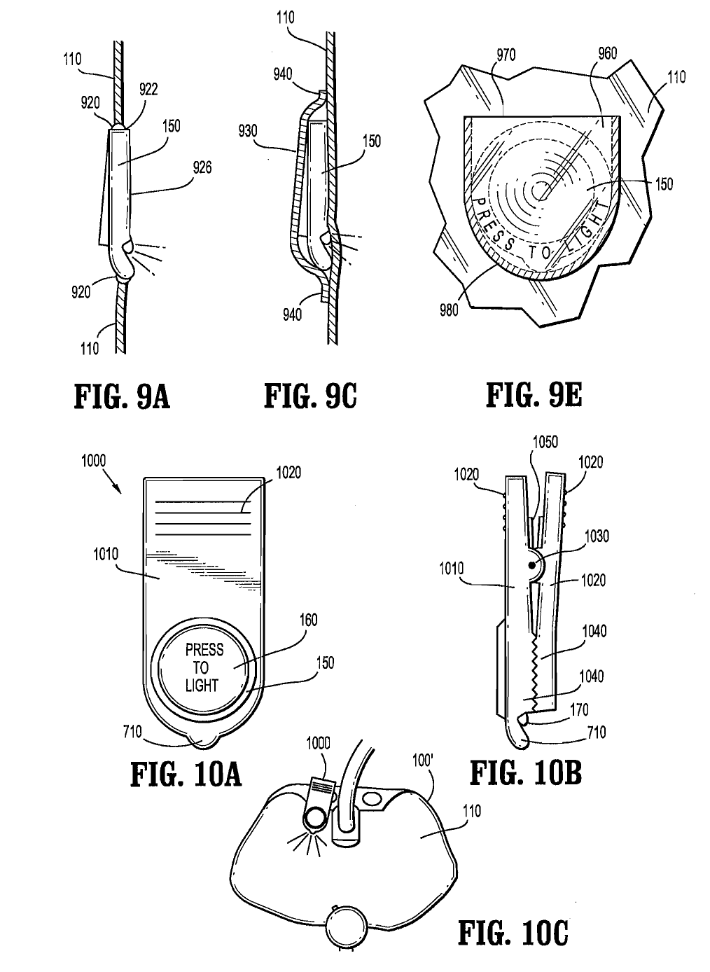

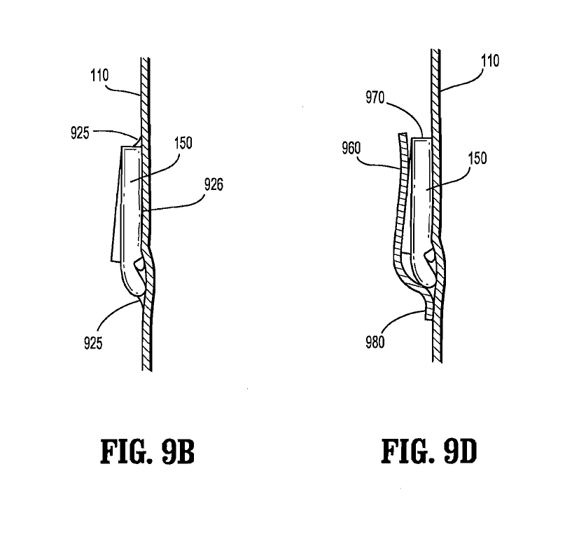

The present disclosure envisions various embodiments, wherein light 150 is affixed to fluid collection bag 100, as will now be described with reference to the figures. FIG. 9A illustrates an embodiment wherein light 150 is substantially permanently mounted to collection bag 100. Aperture 922 is disposed on front panel 110 dimensioned to accept light 150. Light 150 is sealably joined along its edge to front panel 110 by suitable means 920 such as heat welding, chemical welding, or adhesive joining. As can be seen, rear surface 926 of light 150 is in communication with fluid chamber 114, which is desirable, for example, if fluid sensor 850 requires direct fluid contact to sense collected liquid in vessel 114.

In FIG. 9B another embodiment is shown wherein light 150 is substantially permanently joined to the face of front panel 110 by suitable means such as heat welding, chemical welding, or adhesive joining, along the perimeter 925 and/or rear surface 926 of light 150. In yet another embodiment illustrated by FIG. 9C, light 150 is substantially permanently laminated between front panel 110, and laminate cover 930 having at its perimeter a margin 940 that is fixed to front panel 110 by any suitable means, preferably by heat welding.

FIGS. 9D and 9E illustrate two views of an embodiment in which light 150 is removably mounted to bag 110. Pocket 960 having an opening 970 is fixedly joined to front panel 110 at seam 980 by any suitable means, preferably by heat welding. Light 150 is inserted into pocket 960 for use, and can be removed after use. Typically, light 150 will remain in pocket 960 until fluid collection bag 100 is disposed of, at which time light 150 can be removed from pocket 960 and be stored or reused in another bag; or until the at least one battery 310 is exhausted, at which time light 150 can be removed from pocket 960 and replaced with a fresh light 150. Additionally or alternatively, light 150 can be fitted with a fresh at least one battery 310, and re-used.

Turning now to FIGS. 10A-C there is shown an embodiment in accordance with the present disclosure wherein light 150 is integrally included within clip 1000 having a generally clothespin-like structure for mounting on a fluid collection bag 100′. Clip 1000 comprises two opposing members 1010 and 1020 pivotally joined by pivot pin 1030. The opposing members have a finger grip end 1020 and opposing jaw ends 1040. A spring 1050 biases the opposing jaw ends 1050 together to retain clip 1000 on fluid collection bag 100′. The pair of finger grip ends 1020 can be squeezed together to open the opposing jaw ends 1040, clamp 1000 can then be positioned as desired on fluid collection bag 100′, and the finger grip ends released thereby fixing clamp 1000 in place.

It will be understood that various modifications may be made to the embodiments disclosed herein. For example, it is envisioned that the configuration and operation of the disclosed light may be altered in many respects to achieve a variety of different objectives. For example, a light having a plurality of LEDs is envisioned, as well as a light which attaches to a fluid collection bag by self-adhesive means. Further, the present disclosure may be incorporated into devices in other medical and non-medical areas. Therefore, the above description should not be construed as limiting, but merely as exemplifications of preferred embodiments. Those skilled in the art will envision other modifications within the scope and spirit of the claims presented herein.

What is claimed is:

1. A medical fluid collection or distribution system for use in a medical procedure comprising:
a membrane defining a fluid chamber and having at least one fluid port in fluid communication with the fluid chamber for transferring fluids between the fluid chamber and a subject; and
an illuminating assembly mountable with respect to the membrane and having an illuminator adapted to at least partially illuminate the fluid chamber to assist in determining a volume of fluid within the fluid chamber.

2. The medical fluid collection or distribution system in accordance with claim 1, wherein the illuminating assembly is substantially permanently affixed to the membrane

3. The medical fluid collection or distribution system in accordance with claim 1, wherein the illuminating assembly is removably affixed to the membrane.

4. The medical fluid collection or distribution system in accordance with claim 3, further comprising a pocket disposed upon the membrane for retaining the illuminating assembly.

5. The medical fluid collection or distribution system in accordance with claim 1, wherein the illuminating assembly comprises:
a power source operably coupled to a light source; and
a switch for activating the light source.

6. The medical fluid collection or distribution system in accordance with claim 5, wherein the power source comprises at least one battery.

7. The medical fluid collection or distribution system in accordance with claim 5, wherein the light source is a light-emitting diode.

8. The medical fluid collection or distribution system in accordance with claim 5, wherein the switch is a momentary contact, single-pole single-throw pushbutton switch.

9. The medical fluid collection or distribution system in accordance with claim 5, further comprising a controller electrically disposed between the power source, the switch, and the light source, the controller configured to cause the light to be activated in response to activation of the switch.

10. The medical fluid collection or distribution system in accordance with claim 9, wherein the controller is configured to cause the light to be activated in one of momentary-contact mode, push-on/push-off mode, or push-on/push-off mode with a battery-conserving timeout feature.

11. The medical fluid collection or distribution system in accordance with claim 9, wherein the controller is further configured to sense at least one alarm condition and in response thereto cause the light to be activated in a corresponding alarm mode.

12. The light assembly in accordance with claim 11, wherein the alarm condition is indicated by the presence of an excessive volume of fluid in the fluid collection bag.

13. The light assembly in accordance with claim 11, wherein the alarm condition is indicated by the presence of an insufficient volume of fluid in the fluid collection bag.

14. The light assembly in accordance with claim 11, wherein the alarm condition is indicated by a low battery condition.

15. The illuminating assembly in accordance with claim 1, wherein the illuminating assembly includes a light dispersion shroud.

16. The illuminating assembly in accordance with claim 1, wherein the illuminating assembly further comprises a clamp for retaining the illuminating assembly on the fluid collection bag having two opposing members each having a finger grip end and a jaw end;
an orthogonal hinge disposed between the opposing members, the opposing members pivotally joined by the hinge; and
a spring for biasing the opposing jaw ends together to frictionally retain the light assembly on the fluid collection bag.

17. A method of illuminating a fluid collection bag or fluid distribution bag, the method comprising:
affixing a light assembly to the fluid collection bag or fluid distribution bag for illuminating the contents of the fluid collection bag or fluid distribution bag;
providing a means to activate the light assembly; and
illuminating the contents of the fluid collection bag or fluid distribution bag by causing the light to be activated.
